(19) United States
(12) Patent Application Publication
(10) Pub. No.: US 2021/0263006 A1
(43) Pub. Date: Aug. 26, 2021

(54) CONTROLLED MANUFACTURE AND NANO-LEVEL EVALUATION OF KEROGEN-RICH RESERVOIR ROCK

(71) Applicant: Saudi Arabian Oil Company, Dhahran (SA)

(72) Inventors: Katherine Leigh Hull, Houston, TX (US); Younane N. Abousleiman, Norman, OK (US)

(21) Appl. No.: 17/180,416

(22) Filed: Feb. 19, 2021

Related U.S. Application Data
(60) Provisional application No. 62/979,234, filed on Feb. 20, 2020.

(51) Int. Cl. G01N 33/24; G01N 23/04; G01N 23/2251; G01N 23/2206; G01N 3/02; G01N 3/08

(52) U.S. Cl. CPC G01N 33/241; G01N 23/04; G01N 23/2251; E21B 49/02; G01N 3/02; G01N 3/08; G01N 2223/652; G01N 23/2206

(57) ABSTRACT

Controlled manufacture and nano-level evaluation of kerogen-rich reservoir rock can be implemented as a method. A clay mineral found in kerogen-rich shale is selected. An organic component found in kerogen-rich shale is selected. Multiple concentrations of the clay mineral are selected. Multiple concentrations of the organic component are selected. Multiple kerogen-rich shale samples are fabricated. Each sample includes a first concentration of the multiple concentrations of the clay mineral and a second concentration of the multiple concentrations of the organic component. A microscale beam is formed of each fabricated sample. A maximum dimension of the microscale beam is at most 100 μm. A mechanical experiment is performed on the microscale beam of each fabricated sample. The mechanical experiment includes a tension test or a compression test. The mechanical experiment on the microscale beam of each fabricated sample is imaged using a scanning electron microscope or a transmission electron microscope. A material parameter of the microscale beam of each fabricated sample is determined based on results of the mechanical experiment and images obtained responsive to the imaging. Effects of the clay mineral on the kerogen-rich shale are determined based on the material parameter of the microscale beam of each fabricated sample.

### CROSS-REFERENCE TO RELATED APPLICATIONS

This application claims the benefit of U.S. Application Ser. No. 62/979,234 filed on Feb. 20, 2020, the entire contents of which are incorporated by reference in its entirety.

### TECHNICAL FIELD

This disclosure relates to evaluating rocks, for example, kerogen-rich reservoir rock.

### BACKGROUND

Unconventional source rock reservoirs are formations that contain the hydrocarbon source material interwoven in the rock matrix of silicates, clays, carbonates, etc. along with trapped hydrocarbons (for example, oil, natural gas, or combinations of them). The chemical, physical, and mechanical properties of this complex organic and inorganic porous granular material are complex and difficult to establish. Extraction of hydrocarbons from such reservoirs typically involves increasing the mobility of the hydrocarbons, for example, by hydraulic fracturing. In hydraulic fracturing, a fracturing fluid (loaded with proppants and one or more chemicals in an aqueous or non-aqueous base fluid) is flowed through the hydrocarbon reservoir. The fracturing fluid fractures the reservoir rock to increase mobility of the trapped hydrocarbons.

### SUMMARY

This disclosure relates to controlled manufacture and nano-level evaluation of kerogen-rich reservoir rock. The techniques described in this disclosure can be implemented in a laboratory environment with a goal of recreating in situ reservoir rock and testing such rock in a controlled environment with a goal of understanding the behavior of such rock in situ.

Certain aspects of the subject matter described here can be implemented as a method. A clay mineral found in kerogen-rich shale is selected. An organic component found in kerogen-rich shale is selected. Multiple concentrations of the clay mineral are selected. Multiple concentrations of the organic component are selected. Multiple kerogen-rich shale samples are fabricated. Each sample includes a first concentration of the multiple concentrations of the clay mineral and a second concentration of the multiple concentrations of the organic component. A microscale beam is formed of each fabricated sample. A maximum dimension of the microscale beam is at most 100 μm. A mechanical experiment is performed on the microscale beam of each fabricated sample. The mechanical experiment includes a tension test or a compression test. The mechanical experiment on the microscale beam of each fabricated sample is imaged using a scanning electron microscope or a transmission electron microscope. A material parameter of the microscale beam of each fabricated sample is determined based on results of the mechanical experiment and images obtained responsive to the imaging. Effects of the clay mineral on the kerogen-rich shale are determined based on the material parameter of the microscale beam of each fabricated sample.

An aspect combinable with any other aspect includes the following features. Effects of the organic component on the kerogen-rich shale are determined based on the material parameter of the microscale beam of each fabricated sample.

An aspect combinable with any other aspect includes the following features. The clay mineral is a first clay mineral. The multiple kerogen-rich shale samples are multiple first kerogen-rich shale samples. The material parameter is a first material parameter. A second clay mineral found in kerogen-rich shale is selected. The second clay mineral is different from the first clay window. Multiple concentrations of the second clay mineral are selected. Multiple second kerogen-rich shale samples are fabricated. Each second sample includes a third concentration of the multiple concentrations of the second clay mineral and a fourth concentration of the multiple concentrations of the organic component. A microscale beam is formed of each fabricated second sample. A maximum dimension of the microscale beam is at most 1000 μm. The mechanical experiment is performed on the microscale beam of each fabricated second sample. The mechanical experiment on the microscale beam of each fabricated second sample is imaged using a scanning electron microscope or a transmission electron microscope. A second material parameter of the microscale beam of each fabricated second sample is determined based on the results of the mechanical experiment and images obtained responsive to the imaging. Effects of the second clay mineral on the kerogen-rich shale are determined based on the material parameter of the microscale beam of each fabricated second sample.

An aspect combinable with any other aspect includes the following features. Multiple third kerogen-rich shale samples are fabricated. Each third sample includes a fifth concentration of the multiple concentrations of the first clay mineral, a sixth concentration of the multiple concentrations of the second clay mineral and a seventh concentration of the multiple concentrations of the organic component. A microscale beam is formed of each fabricated third sample. A maximum dimension of the microscale beam is at most 1000 μm. The mechanical experiment is performed on the microscale beam of each fabricated third sample. The mechanical experiment on the microscale beam of each fabricated third sample is imaged using a scanning electron microscope or a transmission electron microscope. The third material parameter of the microscale beam of each fabricated third sample is determined based on results of the mechanical experiment and images obtained responsive to the imaging. Effects of the first clay mineral and the second clay mineral on the kerogen-rich shale are determined based on the material parameter of the microscale beam of each fabricated third sample.

An aspect combinable with any of the other aspects includes the following features. The microscale beam is a cantilever beam.

An aspect combinable with any of the other aspects includes the following features. The microscale beam is a pillar.

An aspect combinable with any of the other aspects includes the following features. The material parameter is a Young's Modulus of the microscale beam.

An aspect combinable with any of the other aspects includes the following features. The material parameter is a modulus of rupture of the microscale beam.

An aspect combinable with any of the other aspects includes the following features. The material parameter is a tensile strength of the microscale beam.

An aspect combinable with any of the other aspects includes the following features. The material parameter is a compressive strength of the microscale beam.

An aspect combinable with any of the other aspects includes the following features. Multiple material parameters of the multiple fabricated samples are stored in a computer-readable storage medium.

An aspect combinable with any of the other aspects includes the following features. The material parameter of the microscale beam is determined by implementing machine-learning algorithms on the multiple material parameters.

An aspect combinable with any of the other aspects includes the following features. The mechanical experiment is a tension test.

An aspect combinable with any of the other aspects includes the following features. The mechanical experiment is a cantilever test.

An aspect combinable with any of the other aspects includes the following features. The mechanical experiment is a compression test.

An aspect combinable with any of the other aspects includes the following features. To image the mechanical experiment, multiple images of the microscale beam at different time instances during the mechanical experiment are captured.

An aspect combinable with any of the other aspects includes the following features. The microscale beam includes multiple stack shale bedding planes. The mechanical experiment on the microscale beam is performed either parallel to order perpendicular to the multiple stack shale bedding planes.

Certain aspects of the subject matter described here can be implemented as a method. Multiple first kerogen-rich shale samples are fabricated. Each first shale sample includes differing first concentrations of a first clay mineral and differing second concentrations of an organic component found in kerogen-rich shale. Multiple microscale beams are formed of each first sample. A maximum dimension of the microscale beam is at most 100 μm. A mechanical experiment is performed on the microscale beam of each fabricated first sample. The mechanical experiment includes a tension test or a compression test. The mechanical experiment on the microscale beam of each fabricated first sample is imaged using a scanning electron microscope or a transmission electron microscope. A material parameter of the microscale beam of each fabricated first sample is determined based on results of the mechanical experiment and images obtained responsive to the imaging. Effects of the differing first concentrations of the first clay mineral on differing concentrations of the organic component are determined based on the material parameter of the microscale beam of each fabricated first sample.

An aspect combinable with any other aspect includes the following features. Multiple second kerogen-rich shale samples are fabricated. Each second shale sample includes differing second concentrations of a second clay mineral and the differing concentrations of the organic component. Multiple microscale beams are formed of each second shale sample. A maximum dimension of the microscale beam is at most 100 μm. The mechanical experiment is performed on the microscale beam of each fabricated second sample. The mechanical experiment on the microscale beam of each fabricated second sample is imaged using the scanning electron microscope or the transmission electron microscope. A material parameter of the microscale beam of each fabricated second sample is determined based on results of the mechanical experiment performed on and images obtained responsive to the imaging of the multiple microscale beams of each second sample. Effects of the differing second concentrations of the second clay mineral on the differing concentrations of the organic component are determined based on the material parameter of the microscale beam of each fabricated second sample.

An aspect combinable with any other aspect includes the following features. Multiple third kerogen-rich shale samples are fabricated. Each third shale sample includes the differing first concentrations of the first clay mineral, the differing second concentrations of the second clay mineral and the differing concentrations of the organic component. Multiple microscale beams of each third sample are formed. A maximum dimension of the microscale beam is at most 100 μm. The mechanical experiment is performed on the microscale beam of each fabricated second sample. The mechanical experiment on the microscale beam of each fabricated third sample is imaged using the scanning electron microscope or the transmission electron microscope. A material parameter of the microscale beam of each fabricated third sample is determined based on results of the mechanical experiment performed on and images obtained responsive to the imaging of the multiple microscale beams of each third sample. Effects of the differing first concentrations of the first clay mineral and the differing concentrations of the second clay mineral on the differing concentrations of the organic component are determined based on the material parameter of the microscale beam of each fabricated third sample.

An aspect combinable with any other aspect includes the following features. The material parameter of the microscale beam of each fabricated first sample, each fabricated second sample and each fabricated third sample are stored in a computer-readable storage medium.

An aspect combinable with any of the other aspects includes the following features. The effects of the differing first concentrations of the first clay mineral on differing concentrations of the organic component are determined by implementing machine-learning algorithms on material parameters stored in the computer-readable storage medium.

An aspect combinable with any of the other aspects includes the following features. The effects of the differing second concentration of the second clay mineral on differing concentrations of the organic component are determined by implementing machine-learning algorithms on material parameters stored in the computer-readable storage medium.

An aspect combinable with any of the other aspects includes the following features. The effects of the differing first concentrations of the first clay mineral and the effects of the differing second concentrations of the second clay mineral on differing concentrations of the organic component are determined by implementing machine-learning algorithms on material parameters stored in the computer-readable storage medium.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic diagram of forming multiple shale samples and evaluating the same based on nano-indentation testing.

FIGS. 2A-2C are flowcharts of a process of forming multiple shale samples and evaluating the same based on nano-indentation testing.

FIG. 3 is a schematic diagram of a fracture treatment for a well.

Like reference, numbers and designations in the various drawings indicate like elements.

### DETAILED DESCRIPTION

All shale source rock reservoirs have the major components of non-clay minerals like quartz, feldspar, and plagioclase, QFP, clays such as illite, mica, smectite, and finally organic matter such as kerogen, and bitumen where the oil and gas reside. An unconventional shale reservoir with 5 wt. % kerogen (˜10 vol. %) is considered kerogen rich. In this disclosure, all the various types of organic matter described above are considered to be components of kerogen, since what is of interest is the mechanics of failure of the composite organic-rich shale, and not the stage of maturity of the organic matter or the reservoir potentials. In this nano-/micro-mechanics approach, the isolated contribution of each kerogen-rich shale (KRS) component and the role it plays in the intertwined phenomena of minerals and kerogen matrices and the different mechanisms of failure were observed. This specification describes interpretations of the experimental results and provides a preliminary numerical model based on the likely percent weight that the interlaced polymer kerogen contributes to the overall shale sample behavior

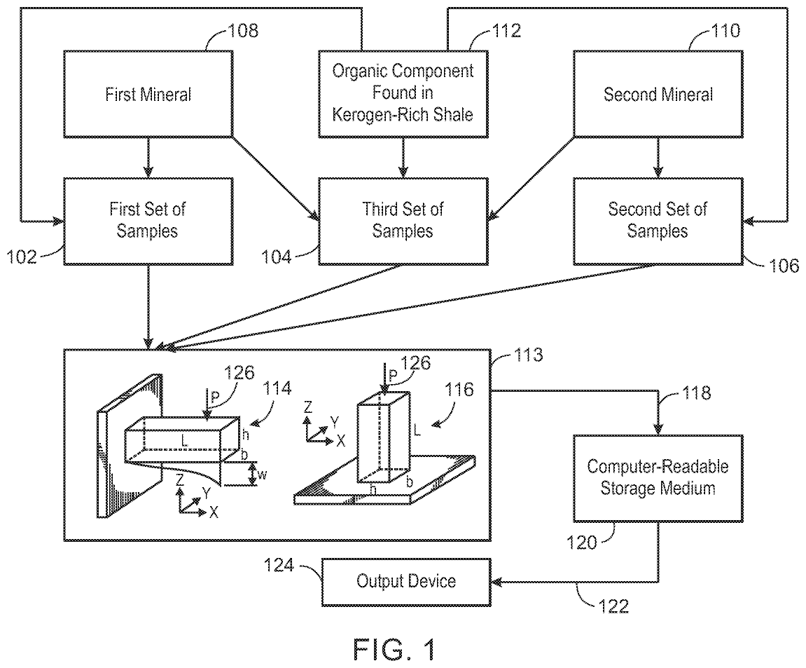

FIG. 1 is a schematic diagram of forming multiple shale samples and evaluating the same based on nano-indentation testing. The schematic diagram presents a bottom-up approach to fabricating shale with different levels of kerogen and mechanically testing multiple shale samples with the goal of reducing uncertainty surrounding the shale matrix. By implementing the techniques described in this disclosure, a large ensemble of manufactured shale samples 102, 104, and 106 are prepared in a laboratory using known pure clay and non-clay mineral sources 108 and 110 in pre-determined ratios and with pre-determined amounts or types (or both) of the organic matter 112. In this manner, the shale preparation is fully controlled in mass fraction or volume fraction (or both), thereby reducing the uncertainty associated with components in a sample, amounts in which the components are present, spatial distribution and the like. The manufactured shale samples 102, 104, and 106 are then cut and shaped into microscale beams, or microbeams 113, forming cantilevers 114 or pillars 116, for example, using focused ion beams (FIB), with controlled and preferred composition and orientation. The samples 114 and 116 are then tested 118 using nano-scale mechanical tests and imaged using a scanning electron microscope (SEM) or a transmission electron microscope (TEM) as described in this disclosure. The mechanical parameters obtained are stored in a computer-readable storage medium 120. Machine learning algorithms are applied 122 to the parameters stored in the computer-readable storage medium 120 to determine the effects of the concentration changes. The effects of the modeling are provided to an output device 124 for user actions. The output device 124 may include a monitor or printer, among others. An input device may be used to determine the next actions, such as providing the model to a reservoir simulation, among others.

In some implementations, individual components of source shale samples, for example, for use as the first mineral 108, or the second mineral 110, or both, are identified. Source shale is a heterogeneous mud rock with a wide range of minerals. The percentage of each component varies from one shale to the next. Some typical components in source rock shale include silicates such as quartz, mica, feldspar (10-60 wt. %), clays such as illite, smectite, kaolinite, montmorillonite (30-70 wt. %, typically 50-60 wt. %), pyrite (1-20 wt. %), carbonates such as calcite, dolomite, siderite (<10 wt. %), organic matter such as kerogen, bitumen and pyrobitumen (2-20 wt. %, average 8-12 wt. %). Source rocks can alternatively be predominantly carbonate with similar contents as source shale and minor amounts of clays, silicates, pyrite. Any combination of components can be selected to form a particular type of sample. For example, kerogen rich shale (KRS) is one type of sample while kerogen-free shale (KFS) is another type of sample. KRS can be further divided into different types, for example, a type that includes a first mineral 108 and an organic component 112, a type that includes a second mineral 110 and the organic component 112, a type that includes the first and second clay minerals 108 and 110 and the organic component 112, to name a few. In this manner, multiple types of shale samples 102, 104, and 106 can be formed using the individual components 108, 110, and 112 of the shale.

Within each type of sample 102, 104, and 106, multiple sub-types can be formed by varying the concentration of each component. For example, the sample type that includes the first mineral 108 and the organic component 112 can be divided into multiple sub-types, each sub-type including a concentration of either the first mineral 108 or the organic component 112. In one example, different sub-types can be formed by fixing the concentration of the first mineral 108 and step-wise increasing the concentration of the organic component 112. Additional sub-types can be formed by similarly varying the concentration of the first mineral 108 and fixing the concentration of the organic component 112. By repeating this procedure across different types of samples, a multi-dimensional array of types of shale samples 102, 104, and 106, each type further divided into a multi-dimensional array of subtypes of shale samples, can be formed.

Example techniques for forming a sample are described here. In the schematic shown in FIG. 1, three different sets of samples 102, 104, and 106 are formed. These include a first set of samples 102 that include the first mineral 108 and the organic component 112, a second set of samples that include the second mineral 110 and the organic component 112, and a third set of samples that include the first and second clay minerals 108 and 110 and the organic component 112.

In the next step, test samples 114 and 116 for nano-evaluation are fabricated from each sub-type. As described herein, each test sample 114 and 116 can be a microbeam 113, that is, a microscale beam with a maximum dimension of at most 1000 micrometer (μm). The microbeams 113 can be formed as cantilevers 114 or pillars 116.

In some implementations, each cantilever 114 can have dimensions of 1×1×0.4 cm. The cantilever 114 can be cut from one of the samples formed using the techniques described earlier. For example, a sharp 90° edge can created by mechanical polishing using standard silicon carbide paper up to 4000 grit followed by polishing with 1 μm diamond grit. A Quanta 3D field emission gun (FEG) with FIB-SEM can be used to prepare the cantilever 114. FIB surface milling can be used to clean the surface for better sample imaging as well as to prepare the desired micro-geometries. The cantilever 114 can be fabricated using the FIB procedure according to the S. G. Roberts method (Maio and Roberts 2005, Frazer et al. 2015). Each cantilever 114 can be shaped by cutting trenches on all three sides of a sample with widths of 20 μm and depths of 10 μm using a 15 nA beam current, resulting in a U-shaped trench. The geometry can then be refined by applying a 1 nA beam current. Afterwards, the sample can be tilted to 45° along the length axis to shape the cantilever 114. The base of the cantilever 114 can be undercut from both sides using a 3 nA beam current, forming the final shape of the cantilever 114.

In some implementations, each pillar 116 can be manufactured using the FIB instrument described earlier to be a square micro-pillar with minimal taper. The FIB instrument can be implemented with successively lower beam currents (5 nA down to 0.3 nA at 30 kV). Alternatively, the micro-pillars can have other cross-sectional shapes, for example, round cross-section. The milling procedure can follow the methods of earlier work (Maio and Roberts 2005; Hosemann et al. 2008 and 2013; Shin et al. 2014). To achieve the square geometry, the sample can be tilted by ±2° with respect to the incident ion beam in order to mill the side surfaces of the pillar by grazing incident ions. The aspect ratio (micro-pillar height divided by width) can be set close to three to one as in Hosemann et al. (2008). These dimensions may vary slightly, eventually, if these tests are to be standardized for porous natural material such as shale.

By implementing the fabrication techniques described earlier, multiple cantilevers 114 or multiple pillars 116 or both can be fabricated for each sub-type of each type of shale sample. The array of microbeams 113, each having different, but known and predetermined components, can then be tested 118 to develop a comprehensive repository of mechanical parameters in the computer-readable storage medium 120 that can be evaluated to understand the effect of each concentration of each component 108, 110, or 112 or any combination of any concentration of any number of components 108, 110, and 112 on the mechanical properties of shale and the effect of kerogen on the mechanical properties.

In some implementations, the mechanical experiments performed on each of the microbeams 113 include nano-indentation experiments to measure displacements across different nano-indentation loads. The experiments can be imaged, for example, using a SEM or TEM, while the experiments are being performed. For example, a Hysitron Pi-85 Pico-indenter can be used to load the microbeams 113 under displacement control mode, at a rate of 10 nm/s. The indenter tip is a flat circular punch geometry, with a diameter of 5 μm. All loading experiments can be performed in situ under the SEM, where loading of the cantilevers 114 continued until failure. For example, the indenter tip can be placed at the end of the microbeams 113, centered along the z-axis as shown in FIG. 1 (other mapping of the axes may be used in the SEM). Load and displacement data can be collected in real-time.

During the experiment, a force 126 (in micro-Newtons) is applied to the cantilever 114 or pillar 116 through the nanoindenter tip. As the force 126 is applied, the cantilever 114 or pillar 116 deforms (meaning the indenter tip is displaced in nanometers). Both the force 126 and displacement are captured by the nanoindenter software throughout the experiment. Typically the rate of displacement is controlled (for example, 10 nm/second), while the force is applied to such a degree as to maintain this displacement rate. Because this experiment is performed inside a scanning electron microscope (SEM), the fourth parameter captured (beyond force, displacement, and time) is an SEM image. In fact, the SEM images are captured throughout the entire loading experiment, as a movie of the entire experiment. Finally, additional analysis of the cantilever 114 or pillar 116 can also be performed with energy dispersive x-ray spectroscopy (EDS) while the sample is inside the SEM. This measurement provides the chemical (elemental) composition of the sample. It can be performed pre-loading, post-failure, or in some configurations, during the loading.

By implementing the mechanical experiments described herein, mechanical parameters for multiple samples of each sub-type of each type of shale sample 102, 104, or 106 can be measured. The mechanical parameters, each obtained from samples having different, but known and predetermined components, can then be evaluated to develop a comprehensive understanding of the effect of each concentration of each component 108, 110, or 112 or any combination of any concentration of any number of components 108, 110, and 112 on the mechanical properties of shale and the effect of kerogen on the mechanical properties.

In some implementations, the mechanical parameters measured by implementing the techniques described here can be stored on the computer-readable storage medium 120, for example, in a computer database. Data analytics can be implemented on the stored mechanical parameters to draw inferences and understanding about the effect of each component on the overall mechanical properties of shale samples. In some implementations, machine-learning algorithms are applied 122 to the data for analysis.

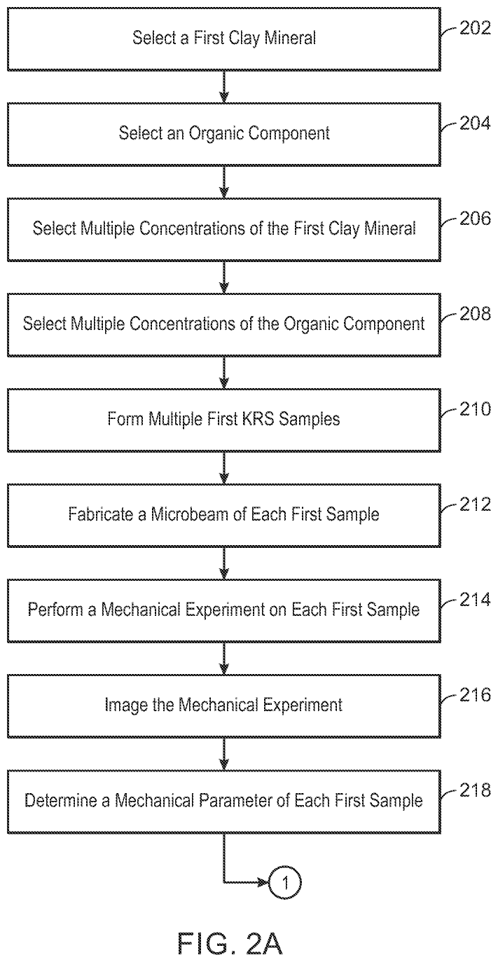

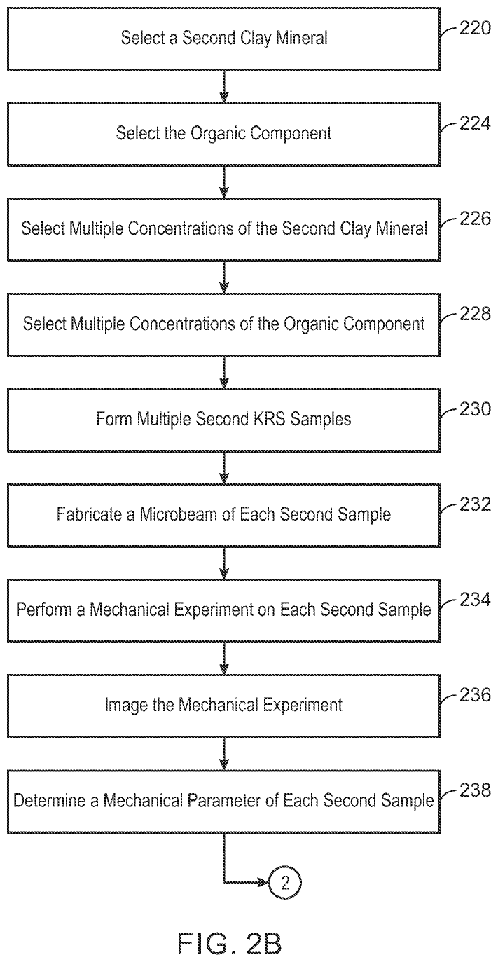

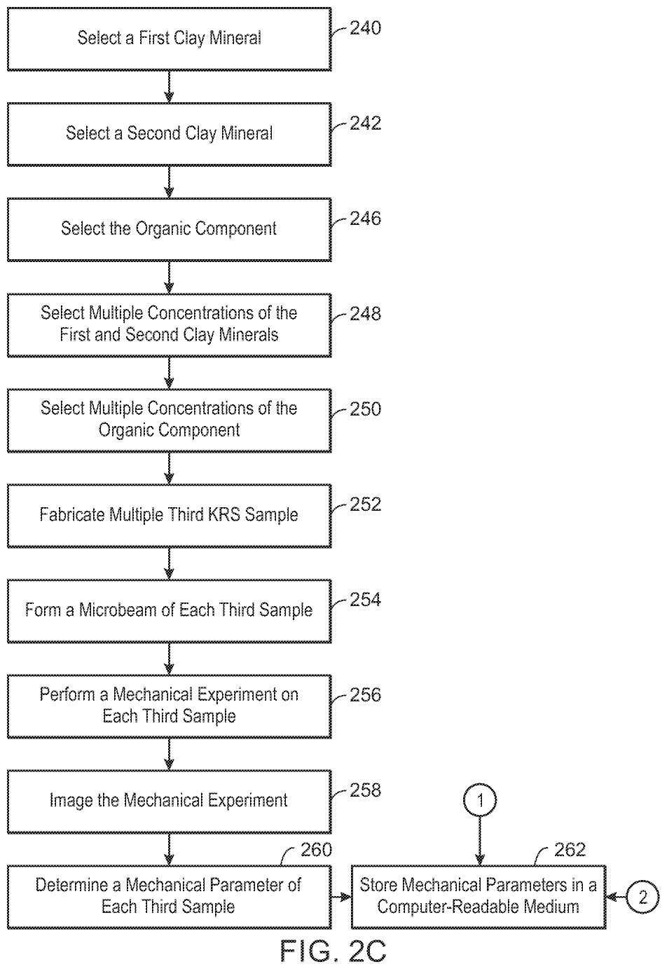

FIGS. 2A-2C are flowcharts of a process of forming multiple shale samples and evaluating the same based on nano-indentation testing. In the example process shown in FIGS. 2A-2C, three components—a first clay mineral, a second clay mineral, an organic component found in kerogen-rich shale—are selected to form three types of samples—the first type, shown in FIG. 2A, including the first clay mineral and the organic component, the second type, shown in FIG. 2B, including the second clay mineral and the organic component, and the third type, shown in FIG. 2C, including both clay minerals and the organic component. The process can be implemented with additional components including, for example, other clay minerals, other organic components, to name a few. The process can also be implemented to form KFS samples and to evaluate such samples using the same techniques described with reference to KRS samples.

In some implementations, as shown in FIG. 2A, the first clay mineral is selected (block 202) and the organic component is selected (block 204). Multiple concentrations of each of the first clay mineral (block 206) and the organic component are selected (block 208). Multiple first KRS samples are formed (block 210), each having a different concentration of the first clay mineral or the organic component (or both) compared to another first KRS sample. A microbeam (for example, a cantilever or a pillar) is fabricated (block 212) from each first KRS sample. Mechanical experiments are performed (block 214) on each first KRS sample. The mechanical experiments include the nano-indentation experiments described earlier. The mechanical experiments can be imaged (block 216), for example, using a SEM or a TEM, while the experiments are being performed. Mechanical parameters can be determined (block 218) for each first KRS sample. The mechanical parameters can include, for example, Young's modulus, Modulus of rupture, tensile strength, compressive strength, to name a few.

The process steps described in the preceding paragraph can be repeated (blocks 220-238) for the second KRS sample as shown in FIG. 2B. Similar steps may be performed for the third KRS sample (blocks 240-260), as shown in FIG. 2C. In the process of FIG. 2C an extra component, a second clay mineral, is selected for the samples, as indicated at block 242. Further KRS samples may be formed and tested as well. The mechanical parameters determined for each sample can be stored (block 262) in a computer-readable storage medium to form a database collection of mechanical parameters of different types of shale parameters.

In some implementations, machine learning algorithms or data analytics (or both) can be applied to the data gathered in the database. The output of the machine learning algorithms or the data analytics (or both) can be the effects of individual components or groups of components on the behavior of kerogen in kerogen-rich shale. The information obtained from such analysis can be used to improve hydraulic fracturing processes such as those described later. In some implementations, the experiments described here can be performed with a nano-indenter heating stage inside the imaging equipment. By doing so, the variation in mechanical parameters with change in temperature can be captured. Such variation can provide a more accurate mechanical property profile for the components of the samples in the downhole environment. In some implementations, the anisotropy of each sample can be controlled by varying the magnitude of confining stresses used to compact the components to form the sample. Doing so can control the matrix anisotropy. In some implementations, KRS samples formed using the techniques described here can be treated with a fluid that breaks down kerogen in the sample before forming the microbeams. After treating the samples with the fluid, the mechanical experiments described in this disclosure can be performed. Doing so can provide information on an effect of the fluid on the kerogen in the shale sample.

Example of a Hydraulic Fracture Treatment Process

The experiments discussed prior can yield valuable data. For example, the fracturability of mudstone can be predicted by interpreting the load curves from varying samples. The fracturability data assists in calculated pressure in flow rates during a hydraulic fracture treatment process, such as the example illustrated later. The experiments discussed prior can also be utilized for evaluating different chemical treatments. For example, a shale sample can be treated with a fluid designed to break-down kerogen. The treated sample can then be fabricated into a microbeam and tested to demonstrate the fluids effects on kerogen. Such knowledge can improve the effectiveness of hydraulic fracture treatments such as the example given in the following paragraphs.

The kerogen content of different microbeam specimens in the previously discussed experiments can be varied and the tensile test results compared directly. The microbeam specimen can even come from the same bulk shale sample, but taken from high, low, or intermediate kerogen content regions. Without the kerogen, the microbeam will undergo brittle tensile failure under load, with minimal tensile mode energy required to break it. With kerogen, the energy required as well as its correlative tensile strength will be much higher.

In compression, higher kerogen content will lead to lower compressive strength. Therefore, two pillars of equivalent size and dimension but different kerogen content will yield differently under compressive loads. Kerogen is understood to be at least 10s time weaker than the rock granular structure, depending on its maturity, in compression. Hydraulic fracturing is primarily a tensile failure of the rock in a Mode I fracture propagation criteria, so the tensile properties (in cantilever tests) are the most relevant to fracturability considerations when it comes to optimizing hydraulic fracturing planning and execution

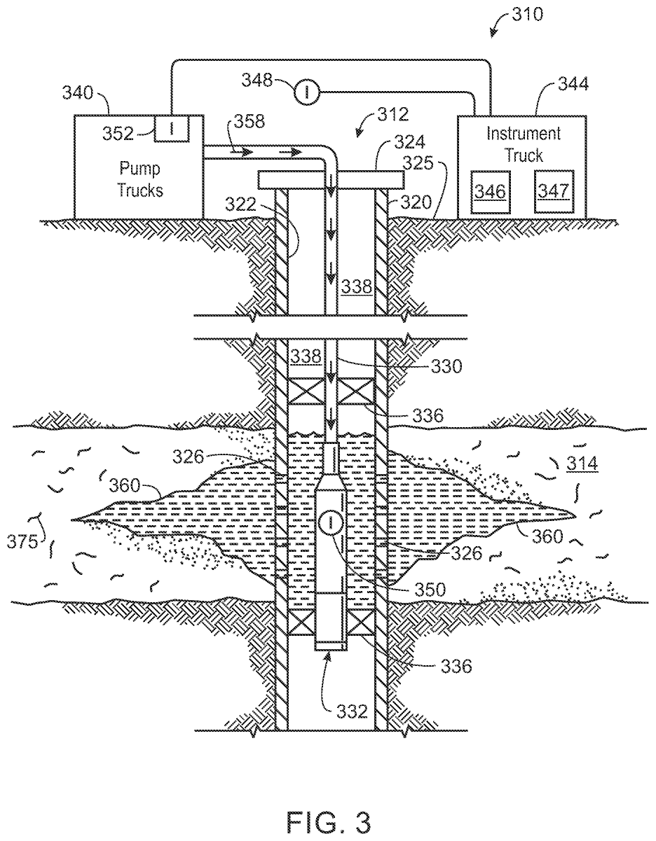

FIG. 3 is a schematic diagram of a fracture treatment 310 for a well 312. The well 312 can be a reservoir or formation 314, for example, an unconventional reservoir in which recovery operations in addition to conventional recovery operations are practiced to recover trapped hydrocarbons. Examples of unconventional reservoirs include tight-gas sands, gas and oil shales, coalbed methane, heavy oil and tar sands, gas-hydrate deposits, to name a few. In some implementations, the formation 314 includes an underground formation of naturally fractured rock containing hydrocarbons (for example, oil, gas or both). For example, the formation 314 can include a fractured shale. In some implementations, the well 312 can intersect other suitable types of formations 314, including reservoirs that are not naturally fractured in any significant amount.

The well 312 can include a well bore 320, casing 322, and wellhead 324. The well bore 320 can be a vertical or deviated bore. The casing 322 can be cemented or otherwise suitably secured in the well bore 312. Perforations 326 can be formed in the casing 322 at the level of the formation 314 to allow oil, gas, and by-products to flow into the well 312 and to be produced to the surface 325. Perforations 326 can be formed using shape charges, a perforating gun or otherwise.

For the fracture treatment 310, a work string 30 can be disposed in the well bore 320. The work string 330 can be coiled tubing, sectioned pipe, or other suitable tubing. A fracturing tool 332 can be coupled to an end of the work string 330. Packers 336 can seal an annulus 338 of the well bore 320 above and below the formation 314. Packers 336 can be mechanical, fluid inflatable or other suitable packers.

One or more pump trucks 340 can be coupled to the work string 330 at the surface 325. The pump trucks 340 pump fracture fluid 358 down the work string 330 to perform the fracture treatment 310 and generate the fracture 360. The fracture fluid 358 can include a fluid pad, proppants, and/or a flush fluid. The pump trucks 340 can include mobile vehicles, equipment such as skids, or other suitable structures. The fracturing fluid can be a cross-linked gel, linear gel, synthetic polymer gel, or slickwater with friction reducer. The fluid can be proppant-laden.

One or more instrument trucks 344 can also be provided at the surface 325. The instrument truck 344 can include a fracture control system 346 and a fracture simulator 347. The fracture control system 346 monitors and controls the fracture treatment 310. The fracture control system 346 can control the pump trucks 340 and fluid valves to stop and start the fracture treatment 310 as well as to stop and start the pad phase, proppant phase and/or flush phase of the fracture treatment 310. The fracture control system 346 communicates with surface and/or subsurface instruments to monitor and control the fracture treatment 310. In some implementations, the surface and subsurface instruments may comprise surface sensors 348, down-hole sensors 350, and pump controls 352.

A quantity of energy applied by the fracture control system 346 to generate the fractures 360 in the reservoir or formation 314 can be affected not only by the properties of the reservoir rock in the formation but also by the organic matter (for example, kerogen 375) intertwined within the rock matrix.

Thus, particular implementations of the subject matter have been described. Other implementations are within the scope of the following claims.

What is claimed is:

1. A method comprising:
selecting a clay mineral found in kerogen-rich shale;
selecting an organic component found in kerogen-rich shale;
selecting a plurality of concentrations of the clay mineral;
selecting a plurality of concentrations of the organic component;
fabricating a plurality of kerogen-rich shale samples, each sample comprising a first concentration of the plurality of concentrations of the clay mineral and a second concentration of the plurality of concentrations of the organic component;
forming a microscale beam of each fabricated sample, wherein a maximum dimension of the microscale beam is at most 1000 micrometer (μm);
performing a mechanical experiment on the microscale beam of each fabricated sample, wherein the mechanical experiment comprises a tension test or a compression test;
imaging the mechanical experiment on the microscale beam of each fabricated sample using a scanning electron microscope (SEM) or a transmission electron microscope (TEM);
determining a material parameter of the microscale beam of each fabricated sample based on results of the mechanical experiment and images obtained responsive to the imaging; and
determining effects of the clay mineral on the kerogen-rich shale based on the material parameter of the microscale beam of each fabricated sample.

2. The method of claim 1, further comprising determining effects of the organic component on the kerogen-rich shale based on the material parameter of the microscale beam of each fabricated sample.

3. The method of claim 1, wherein the clay mineral is a first clay mineral, wherein the plurality of kerogen-rich shale samples are a plurality of first kerogen-rich shale samples, wherein the material parameter is a first material parameter, wherein the method further comprises:
selecting a second clay mineral found in kerogen-rich shale, the second clay mineral different from the first clay mineral;
selecting a plurality of concentrations of the second clay mineral;
fabricating a plurality of second kerogen-rich shale samples, each second sample comprising a third concentration of the plurality of concentrations of the second clay mineral and a fourth concentration of the plurality of concentrations of the organic component;
forming a microscale beam of each fabricated second sample, wherein a maximum dimension of the microscale beam is at most 1000 micrometer (μm);
performing the mechanical experiment on the microscale beam of each fabricated second sample;
imaging the mechanical experiment on the microscale beam of each fabricated second sample using a scanning electron microscope (SEM) or a transmission electron microscope (TEM);
determining a second material parameter of the microscale beam of each fabricated second sample based on results of the mechanical experiment and images obtained responsive to the imaging; and
determining effects of the second clay mineral on the kerogen-rich shale based on the material parameter of the microscale beam of each fabricated second sample.

4. The method of claim 3, further comprising:
fabricating a plurality of third kerogen-rich shale samples, each third sample comprising a fifth concentration of the plurality of concentrations of the first clay mineral, a sixth concentration of the plurality of concentrations of the second clay mineral and a seventh concentration of the plurality of concentrations of the organic component;
forming a microscale beam of each fabricated third sample, wherein a maximum dimension of the microscale beam is at most 1000 micrometer (μm);
performing the mechanical experiment on the microscale beam of each fabricated third sample;
imaging the mechanical experiment on the microscale beam of each fabricated third sample using a scanning electron microscope (SEM) or a transmission electron microscope (TEM);
determining a third material parameter of the microscale beam of each fabricated third sample based on results of the mechanical experiment and images obtained responsive to the imaging; and
determining effects of the first clay mineral and the second clay mineral on the kerogen-rich shale based on the material parameter of the microscale beam of each fabricated third sample.

5. The method of claim 1, wherein the microscale beam is a cantilever beam.

6. The method of claim 1, wherein the microscale beam is a pillar.

7. The method of claim 1, wherein the material parameter is a Young's Modulus of the microscale beam.

8. The method of claim 1, wherein the material parameter is a modulus of rupture of the microscale beam.

9. The method of claim 1, wherein the material parameter is a tensile strength of the microscale beam.

10. The method of claim 1, wherein the material parameter is a compressive strength of the microscale beam.

11. The method of claim 1, further comprising storing a plurality of material parameters of the plurality of fabricated samples in a computer-readable storage medium.

12. The method of claim 11, further comprising determining a material parameter of the microscale beam by implementing machine-learning algorithms on the plurality of material parameters.

13. The method of claim 1, wherein the mechanical experiment is a tension test.

14. The method of claim 1, wherein the mechanical experiment is a cantilever test.

15. The method of claim 1, wherein the mechanical experiment is a compression test.

16. The method of claim 1, wherein imaging the mechanical experiment comprises capturing a plurality of images of the microscale beam at different time instances during the mechanical experiment.

17. The method of claim 1, wherein the microscale beam comprises a plurality of stacked shale bedding planes, wherein the mechanical experiment on the microscale beam is performed either parallel to or perpendicular to the plurality of stacked shale bedding planes.

18. A method comprising:
fabricating a plurality of first kerogen-rich shale samples comprising differing first concentrations of a first clay mineral and differing second concentrations of an organic component found in kerogen-rich shale;
forming a plurality of microscale beams of each first sample, wherein a maximum dimension of the microscale beam is at most 1000 micrometer (μm);
performing a mechanical experiment on each of the plurality of microscale beams of each first sample, wherein the mechanical experiment comprises a tension test or a compression test;
imaging the mechanical experiment on each of the plurality of microscale beams of each first sample using a scanning electron microscope (SEM) or a transmission electron microscope (TEM);
determining a material parameter of each of the plurality of microscale beams of each first sample based on results of the mechanical experiment and images obtained responsive to the imaging; and
determining effects of the differing first concentrations of the first clay mineral on differing concentrations of the organic component based on the material parameter of each of the plurality of microscale beams of each first sample.

19. The method of claim 18, further comprising:
fabricating a plurality of second kerogen-rich shale samples comprising differing second concentrations of a second clay mineral and the differing concentrations of the organic component;
forming a plurality of microscale beams of each second sample, wherein a maximum dimension of the microscale beam is at most 1000 micrometer (μm);
performing the mechanical experiment on each of the plurality of microscale beams of each second sample;
imaging the mechanical experiment on each of the plurality of microscale beams of each second sample using the SEM or the TEM;
determining a material parameter of the microscale beam of each of the plurality of microscale beams of each second sample based on results of the mechanical experiment performed on and images obtained responsive to the imaging of each of the plurality of microscale beams of each second sample; and
determining effects of the differing second concentrations of the second clay mineral on the differing concentrations of the organic component based on the material parameter of each of the plurality of microscale beams of each second sample.

20. The method of claim 19, further comprising:
fabricating a plurality of third kerogen-rich shale samples comprising the differing first concentrations of the first clay mineral, the differing second concentrations of the second clay mineral and the differing concentrations of the organic component;
forming a plurality of microscale beams of each third sample, wherein a maximum dimension of the microscale beam is at most 1000 micrometer (μm);
performing the mechanical experiment on each of the plurality of microscale beams of each third sample;
imaging the mechanical experiment on each of the plurality of microscale beams of each third sample using the SEM or the TEM;
determining a material parameter of each of the plurality of microscale beams of each third sample based on results of the mechanical experiment performed on and images obtained responsive to the imaging of each of the plurality of microscale beams of each third sample; and
determining effects of the differing first concentrations of the first clay mineral and the differing concentrations of the second clay mineral on the differing concentrations of the organic component based on the material parameter of each of the plurality of microscale beams of each third sample.

21. The method of claim 20, further comprising storing, in a computer-readable storage medium, the material parameter of each of the plurality of microscale beams of each first sample, each of the plurality of microscale beams of each second sample, and each of the plurality of microscale beams of each third sample.

22. The method of claim 21, wherein the effects of the differing first concentrations of the first clay mineral on differing concentrations of the organic component are determined by implementing machine-learning algorithms on material parameters stored in the computer-readable storage medium.

23. The method of claim 21, wherein the effects of the differing second concentrations of the second clay mineral on differing concentrations of the organic component are determined by implementing machine-learning algorithms on material parameters stored in the computer-readable storage medium.

24. The method of claim 21, wherein the effects of the differing first concentrations of the first clay mineral and the effects of the differing second concentrations of the second clay mineral on differing concentrations of the organic component are determined by implementing machine-learning algorithms on material parameters stored in the computer-readable storage medium.
